(19) United States
(12) Patent Application Publication
(10) Pub. No.: US 2026/0327744 A1
(43) Pub. Date: Oct. 22, 2026

(54) Method for Recovering Catalyst and Ethylene Carbonate From Liquid Waste Product of Ethylene Carbonate Production

(71) Applicant: LOTTE CHEMICAL CORPORATION, Seoul (KR)

(72) Inventors: Jung Jae LEE, Uiwang-si (KR); So Young KWON, Uiwang-si (KR); Eun Seon KIM, Uiwang-si (KR); Jun Ho CHI, Uiwang-si (KR)

(21) Appl. No.: 19/491,070

(22) PCT Filed: Jun. 7, 2024
(86) PCT No.: PCT/KR2024/007791

§ 371 Date: Dec. 8, 2025

(30) Foreign Application Priority Data
Jun. 14, 2023 (KR) 10-2023-0076232

(51) Int. Cl. C22B 7/00; C07D 317/38; C22B 26/10

(52) U.S. Cl. CPC C22B 7/009; C07D 317/38; C22B 7/005; C22B 26/10

(57) ABSTRACT

A method for recovering a catalyst and ethylene carbonate from a liquid waste product of ethylene carbonate production according to the present invention is characterized by comprising: a step in which a liquid waste product generated in a process for producing ethylene carbonate by reacting ethylene oxide and carbon dioxide in the presence of a metal halide-based catalyst is distilled under reduced pressure to separate and recover ethylene carbonate; a step in which a solid residue remaining after the ethylene carbonate is separated from the liquid waste product is dissolved in dichloromethane and then filtered to remove by-products of the production process; a step in which, after removing the by-products, the filtered solid residue is dissolved in water and then filtered to filter out impurities; and a step for drying the filtrate to recover the metal halide-based catalyst. The recovery method can separate and recover the catalyst and ethylene carbonate at a high yield and purity from the liquid waste product of ethylene carbonate production.

### TECHNICAL FIELD

The present invention relates to a method for recovering a catalyst and ethylene carbonate from an ethylene carbonate production waste liquid. More particularly, the present invention relates to a method that can separate and recover a catalyst and ethylene carbonate from an ethylene carbonate production waste liquid with high yield and high purity.

### BACKGROUND ART

Ethylene carbonate is used as a solvent for various chemical reactions and as an electrolyte solvent in lithium-ion secondary batteries and is a major raw material for polycarbonate resins.

Typically, ethylene carbonate is synthesized by reacting ethylene oxide with carbon dioxide at high temperature and high pressure. Various halide materials can serve as catalysts in this reaction, among which metal halide catalysts are commonly used due to their excellent reactivity and selectivity.

During production of ethylene carbonate, a waste liquid containing ethylene carbonate and a catalyst is produced. Since this waste liquid contains a mixture of various by-products, recycling the liquid directly into the ethylene carbonate production process results in introduction of the by-products into the final ethylene carbonate, leading to quality degradation.

To address these problems, the ethylene carbonate production waste liquid could be disposed of by an external contractor instead of being recycled. However, this approach is disadvantageous from both an economic and environmental standpoint, as it involves disposal costs and leads to waste of valuable substances such as the catalyst and ethylene carbonate contained in the waste liquid.

Therefore, there is a need for a method that can separate and recover a catalyst and ethylene carbonate from an ethylene carbonate production waste liquid with high yield and high purity.

The background technique of the present invention is disclosed in Japanese Patent Publication No. 5762031 and the like.

### DISCLOSURE

### Technical Problem

It is an object of the present invention to provide a method that can separate and recover a catalyst and ethylene carbonate from an ethylene carbonate production waste liquid with high yield and high purity.

The above and other objects of the present invention will become apparent from the detailed description of the following embodiments.

### Technical Solution

1. One aspect of the present invention relates to a method for recovering a catalyst and ethylene carbonate from an ethylene carbonate production waste liquid. The recovery method comprises the steps of: separating and recovering ethylene carbonate by vacuum distillation of a production waste liquid generated during a process of producing ethylene carbonate by reacting ethylene oxide with carbon dioxide in the presence of a metal halide catalyst; removing by-products of the production process by dissolving a solid residue remaining after separation of the ethylene carbonate from the production waste liquid in dichloromethane, followed by filtration; filtering out impurities by dissolving the filtered solid residue remaining after removal of the by-products in water, followed by filtration; and recovering the metal halide catalyst by drying a resulting filtrate.
2. In embodiment 1, the waste liquid comprises about 1 wt % to about 10 wt % of the metal halide catalyst, about 60 wt % to about 95 wt % of the ethylene carbonate, and about 1 wt % to about 30 wt % of the by-products and impurities based on the total weight of the waste liquid.
3. In embodiment 1 or 2, vacuum distillation of the production waste liquid is performed at a temperature of about 100° C. to about 150° C. and at a pressure of about 0.5 torr to about 20 torr.
4. In embodiments 1 to 3, the separated and recovered ethylene carbonate may have a purity of about 99.5% or more, as calculated from an area of a peak detected by a flame ionization detector through a procedure in which a sample, prepared by dissolving the recovered ethylene carbonate in methanol to a concentration of 5 wt %, is injected into a gas chromatograph equipped with a DB-WAX column at an injection temperature of 250° C., followed by maintaining a chamber at a temperature of 40° C. for 5 minutes and raising the temperature of the chamber to 230° C. at a rate of 10° C./min.
5. In embodiments 1 to 4, the dichloromethane is added in an amount of about 80 parts by weight to about 500 parts by weight relative to about 100 parts by weight of the solid residue remaining after separation of the ethylene carbonate.
6. In embodiments 1 to 5, the water is added in an amount of about 80 parts by weight to about 500 parts by weight relative to about 100 parts by weight of the filtered solid residue remaining after removal of the by-products.
7. In embodiments 1 to 6, drying of the filtrate is performed at a temperature of about 60° C. to about 110° C. and at a pressure of 1 torr to about 300 torr.
8. In embodiments 1 to 7, the recovered metal halide catalyst may be free of detectable organic matter, including ethylene carbonate, as measured using a flame ionization detector through a procedure in which a sample, prepared by dissolving the recovered metal halide catalyst in methanol to a concentration of 5 wt %, is injected into a gas chromatograph equipped with a DB-WAX column at an injection temperature of 250° C., followed by maintaining a chamber at a temperature of 40° C. for 5 minutes and raising the temperature of the chamber to 230° C. at a rate of 10° C./min.
9. In embodiments 1 to 8, the method has a recovery rate of about 70% or more for the metal halide catalyst, as calculated according to Equation 1:

Metal halide catalyst recovery rate (%)=(Amount of recovered metal halide catalyst (g)/Amount of metal halide catalyst in ethylene carbonate production waste liquid (g))×100 [Equation 1]
where the amount of the metal halide catalyst in the ethylene carbonate production waste liquid is obtained by multiplying a weight of a waste liquid used by a percentage content (%) of the metal halide catalyst in an entire waste liquid.

### Advantageous Effects

Embodiments of the present invention provide a method that can separate and recover a catalyst and ethylene carbonate from an ethylene carbonate production waste liquid with high yield and high purity.

### BEST MODE

Hereinafter, exemplary embodiments of the present invention will be described in detail.

As used herein to represent a specific numerical range, “a to b” means “a≤ and ≤b”.

The present invention relates to a method for separating and recovering a catalyst and ethylene carbonate from an ethylene carbonate production waste liquid with high yield and high purity, the method comprising the steps of: (A) separating and recovering ethylene carbonate by vacuum distillation of a production waste liquid generated during a process of producing ethylene carbonate by reacting ethylene oxide with carbon dioxide in the presence of a metal halide catalyst; (B) removing by-products of the production process by dissolving a solid residue remaining after separation of the ethylene carbonate from the production waste liquid in dichloromethane, followed by filtration; (C) filtering out impurities by dissolving the filtered solid residue remaining after removal of the by-products in water, followed by filtration; and (D) recovering the metal halide catalyst by drying a resulting filtrate.

In some embodiments, the production waste liquid may have a composition identical to that of a production waste liquid generated during a typical ethylene carbonate production process. For example, the production waste liquid may comprise about 1 wt % to about 10 wt % of the metal halide catalyst, about 60 wt % to about 95 wt % of the ethylene carbonate, and about 1 wt % to about 30 wt % of the by-products and impurities, based on the total weight of the production waste liquid.

In some embodiments, vacuum distillation of the production waste liquid may be performed at a temperature of 100° C. to about 150° C., for example, about 110° C. to about 145° C., and at a pressure of 0.5 torr to about 20 torr, for example, 0.6 torr to about 10 torr, specifically 0.7 torr to about 5 torr, for about 1 hour to about 6 hours. Within these ranges, the ethylene carbonate can be separated and recovered with high yield.

In some embodiments, the separated and recovered ethylene carbonate may have a purity of about 99.5% or more, as calculated from an area of a peak detected by a flame ionization detector through a procedure in which a sample, prepared by dissolving the recovered ethylene carbonate in methanol to a concentration of 5 wt %, is injected into a gas chromatograph equipped with a DB-WAX column at an injection temperature of 250° C., followed by maintaining a chamber at a temperature of 40° C. for 5 min and raising the temperature of the chamber to 230° C. at a rate of 10° C./min.

In some embodiments, the dichloromethane may be added in an amount of about 80 parts by weight to about 500 parts by weight, for example, about 85 parts by weight to about 300 parts by weight, relative to about 100 parts by weight of the solid residue remaining after separation of the ethylene carbonate. Within this range, highly dichloromethane-soluble by-products and residual ethylene carbonate contained in the solid residue can be dissolved in the dichloromethane and can be effectively removed in a liquid phase through filtration along with the dichloromethane.

In some embodiments, the water may be added in an amount of about 80 parts by weight to about 500 parts by weight, for example, about 85 parts by weight to about 300 parts by weight, relative to about 100 parts by weight of the filtered solid residue remaining after removal of the by-products. Within this range, the highly water-soluble metal halide catalyst contained in the solid residue can be selectively dissolved in the water, while water-insoluble impurities (including by-products remaining undissolved in the dichloromethane, and the like) can be effectively filtered out, thus obtaining a filtrate containing the high-purity metal halide catalyst dissolved in the water.

In some embodiments, drying of the filtrate may be performed at a temperature of about 60° C. to about 110° C., for example, about 80° C. to about 105° C., and at a pressure of about 1 torr to about 300 torr, for example, about 5 torr to about 40 torr, for 1 hour to 6 hours. Within these ranges, the metal halide catalyst can be obtained with high yield and high purity while ensuring removal of water from the filtrate.

In some embodiments, the recovered metal halide catalyst may be free of detectable organic matter, including ethylene carbonate, as measured using a flame ionization detector through a procedure in which a sample, prepared by dissolving the recovered metal halide catalyst in methanol to a concentration of 5 wt %, is injected into a gas chromatograph equipped with a DB-WAX column at an injection temperature of 250° C., followed by maintaining a chamber at a temperature of 40° C. for 5 minutes and raising the temperature of the chamber to 230° C. at a rate of 10° C./min.

In some embodiments, the recovery method may have a recovery rate of about 70% or more, for example, about 80% to about 95%, for the metal halide catalyst, as calculated according to Equation 1:

Metal halide catalyst recovery rate (%)={Amount of recovered metal halide catalyst (g)/Amount of metal halide catalyst in ethylene carbonate production waste liquid (g)}×100, [Equation 1]
where the amount of the metal halide catalyst in the ethylene carbonate production waste liquid is obtained by multiplying a weight of a production waste liquid used by a percentage content (%) of the metal halide catalyst in an entire production waste liquid.

Generally, since ethylene carbonate acts as a solvent that dissolves the catalyst, attempting extraction and separation with polar and non-polar solvents without first separating the ethylene carbonate makes it very difficult to completely separate the ethylene carbonate and the catalyst and results in the catalyst being dissolved in both a water layer and the non-polar solvent, causing reduction in recovery rate of the catalyst and ethylene carbonate.

Conversely, in the method for recovering a catalyst and ethylene carbonate according to the present invention, the ethylene carbonate is separated first through vacuum distillation and then the remaining catalyst and impurities are separated based on solubility difference therebetween using a non-polar solvent and water, which results in there being no residual ethylene carbonate during the solubility-based separation step, thereby allowing complete removal of the impurities and thus ensuring recovery of the catalyst with high purity.

As described above, the method for recovering a catalyst and ethylene carbonate from an ethylene carbonate production waste liquid according to the present invention can significantly reduce waste liquid disposal costs and catalyst purchase costs by establishing an eco-friendly process that recovers and recycles the catalyst and ethylene carbonate from the production waste liquid with high yield and high purity.

### MODE FOR INVENTION

Next, the present invention will be described in more detail with reference to some examples. However, it should be noted that these examples are provided for illustration only and are not to be construed in any way as limiting the present invention.

### EXAMPLE

### Example 1

200 g of ethylene carbonate (EC) production waste liquid comprising 2.3 wt % of an alkali metal halide catalyst, 75.3 wt % of ethylene carbonate, and 22.4 wt % of by-products and impurities was subjected to vacuum distillation under conditions of 140° C. and 1 torr for 4 hours, thereby separating a distillate (ethylene carbonate). The separated distillate weighed 147 g. The distillate was dissolved in methanol to a concentration of 5 wt %, thereby preparing a sample, which in turn was injected into a gas chromatograph (GC, model name: GC 7890A, manufacturer: Agilent Technologies Inc.) equipped with a DB-WAX column (product name: J&W DB-WAX, product number: 123-7032, manufacturer: Agilent Technologies Inc.) at an injection temperature of 250° C., followed by maintaining a chamber at a temperature of 40° C. for 5 minutes and raising the temperature of the chamber to 230° C. at a rate of 10° C./min, and then an area of a peak detected by a flame ionization detector was measured, thereby determining the purity of the ethylene carbonate. As a result, the purity of the ethylene carbonate was confirmed to be 99.8%, demonstrating recovery of high-purity ethylene carbonate.

Next, 50 g of dichloromethane (manufacturer: Samchun Pure Chemical Co., Ltd.) was mixed with 53 g of a solid residue remaining after separation of the ethylene carbonate from the production waste liquid, followed by stirring for 1 hour to dissolve the solid residue. Thereafter, dichloromethane and components dissolved therein (including by-products of the production process, and the like) were removed through a filter paper (product name: No. 5C quantitative filter paper, manufacturer: ADVANTEC Co., Ltd.), and then a solid precipitate collected on the filter paper was placed in 50 g of water (distilled water), followed by stirring for 30 minutes to dissolve the precipitate. The resulting solution was filtered through a filter paper (product name: No. 5C quantitative filter paper, manufacturer: ADVANTEC Co., Ltd.) to separate components dissolved in the water while filtering out insoluble impurities, followed by drying the resulting filtrate (water and alkali metal halide catalyst) under conditions of 100° C. and 10 torr for 4 hours, thereby obtaining 4 g of a white solid (alkali metal halide catalyst). The obtained white solid was dissolved in methanol to a concentration of 5 wt % to prepare a sample, which in turn was analyzed using a gas chromatograph (GC, model name: GC 7890A, manufacturer: Agilent Technologies Inc.) equipped with a DB-WAX column (product name: J&W DB-WAX, product number: 123-7032, manufacturer: Agilent Technologies Inc.). As a result, the absence of organic matter, including ethylene carbonate was confirmed. Furthermore, the content of alkali metal and halide components was analyzed using an inductively coupled plasma optical emission spectrometer (ICP-OES, model name: OPTIMA 7300DV, manufacturer: Perkin-Elmer Corporation) and an ion chromatograph (IC, model name: ICS-6000, manufacturer: Thermo Fisher Scientific Co., Ltd., combustion unit: AQF-2100H (manufacturer: Mitsubishi Chemical Corporation)). As a result, it was confirmed that the alkali metal halide catalyst was recovered normally. A recovery rate for the alkali metal halide catalyst was calculated according to Equation 1, and results are shown in Table 1.

### Comparative Example 1

360 g of water and 40 g of dichloromethane were mixed with 200 g of an ethylene carbonate production waste liquid, followed by stirring for 30 minutes, and then the resulting mixture was allowed to stand. As a result, phase separation into a water (polar solvent) layer and a dichloromethane (non-polar solvent) layer was confirmed. The water layer was separated and dried under conditions of 100° C. and 10 torr for 4 hours. Despite this, the resulting product still remained in a liquid phase, with a weight of 110.4 g. Analysis by GC, ICP-OES, and IC revealed a composition of 96.6 wt % ethylene carbonate and 3.4 wt % alkali metal halide catalyst, indicating that a significant amount of ethylene carbonate remained unseparated. A recovery rate for the alkali metal halide catalyst was calculated according to Equation 1, and results are shown in Table 1.

### Comparative Example 2

Another experiment was conducted in the same manner as in Comparative Example 1 except that 200 g of water and 200 g of dichloromethane were mixed with 200 g of an ethylene carbonate production waste liquid. The resulting water layer was separated and dried under conditions of 100° C. and 10 torr for 4 hours. Despite this, the resulting product still remained in a liquid phase, with a weight of 47.8 g. Analysis by GC, ICP-OES, and IC revealed a composition of 93.3 wt % ethylene carbonate and 6.7 wt % alkali metal halide catalyst, indicating that a significant amount of ethylene carbonate remained unseparated. A recovery rate for the alkali metal halide catalyst was calculated according to Equation 1, and results are shown in Table 1.

### Comparative Example 3

A further experiment was conducted in the same manner as in Comparative Example 1 except that 40 g of water and 360 g of dichloromethane were mixed with 200 g of an ethylene carbonate production waste liquid. The resulting water layer was separated and dried under conditions of 100° C. and 10 torr for 4 hours. Despite this, the resulting product still remained in a liquid phase, with a weight of 12.4 g. Analysis by GC, ICP-OES, and IC revealed a composition of 85.1 wt % ethylene carbonate and 14.9 wt % alkali metal halide catalyst, indicating that a significant amount of ethylene carbonate remained unseparated. A recovery rate for the alkali metal halide catalyst was calculated according to Equation 1, and results are shown in Table 1.

### Comparative Example 4

Yet another experiment was conducted in the same manner as in Comparative Example 3. Subsequently, the resulting water layer was separated and subjected to vacuum distillation under conditions of 140° C. and 1 torr for 4 hours. Despite this, the resulting product was present as a mixture of solid and liquid phases, with a weight of 2.3 g. Analysis by GC, ICP-OES, and IC revealed a composition of 21.5 wt % ethylene carbonate and 78.5 wt % alkali metal halide catalyst, indicating that, although the catalyst content was high, a significant amount of ethylene carbonate still remained unseparated. A recovery rate for the alkali metal halide catalyst was calculated according to Equation 1, and results are shown in Table 1.

### Property Evaluation

Metal halide catalyst recovery rate (unit: %): A recovery rate for a metal halide catalyst was calculated according to Equation 1:

Metal halide catalyst recovery rate (%)=(Amount of recovered metal halide catalyst (g)/Amount of metal halide catalyst in ethylene carbonate production waste liquid (g))×100 [Equation 1]

where the amount of the metal halide catalyst in the ethylene carbonate production waste liquid is obtained by multiplying a weight of a production waste liquid used by a percentage content (%) of the metal halide catalyst in an entire production waste liquid.

For reference, the amount of the recovered alkali metal halide catalyst in Comparative Examples 1 to 4 was calculated based on the content determined through analysis by GC, ICP-OES, and IC on the resulting products (in a liquid phase or a liquid/solid mixed phase) obtained in Comparative Examples 1 to 4.

TABLE 1

| | Example | Comparative Example | | | |
|---|---|---|---|---|---|
| | 1 | 1 | 2 | 3 | 4 |
| Weight of resulting | 4.0 | 110.4 | 47.8 | 12.4 | 2.3 |
| recovered product (g) | | | | | |
| Catalyst content (wt %) | 100 | 3.4 | 6.7 | 14.9 | 78.5 |
| EC content (wt %) | 0 | 96.6 | 93.3 | 85.1 | 21.5 |
| Catalyst recovery rate (%) | 87.0 | 81.6 | 69.6 | 40.2 | 39.3 |

From the above results, it can be seen that the method for recovering a catalyst and ethylene carbonate from an ethylene carbonate production waste liquid according to the present invention could separate and recover both a metal halide catalyst and ethylene carbonate with high yield and high purity by first separating and recovering ethylene carbonate from the production waste liquid through vacuum distillation before proceeding to separate and recover the metal halide catalyst.

Conversely, it can be seen that the recovery methods according to Comparative Examples, in which ethylene carbonate was not first separated and recovered, resulted in there being an excessive amount of unseparated ethylene carbonate in the resulting product along with dichloromethane, and failed to recover the metal halide catalyst with high yield.

Although some embodiments have been described herein, it will be understood by those skilled in the art that various modifications, changes, and alterations can be made without departing from the spirit and scope of the invention. Therefore, it should be understood that these embodiments are provided for illustration only and are not to be construed in any way as limiting the present invention. The scope of the present invention should be defined by the appended claims rather than by the foregoing description, and the claims and equivalents thereto are intended to cover such modifications and the like as would fall within the scope of the invention.

1. A method for recovering a catalyst and ethylene carbonate from an ethylene carbonate production waste liquid, the method comprising:
separating and recovering ethylene carbonate by vacuum distillation of a production waste liquid generated during a process of producing ethylene carbonate by reacting ethylene oxide with carbon dioxide in the presence of a metal halide catalyst;
removing by-products of the production process by dissolving a solid residue remaining after separation of the ethylene carbonate from the production waste liquid in dichloromethane, followed by filtration;
filtering out impurities by dissolving the filtered solid residue remaining after removal of the by-products in water, followed by filtration; and
recovering the metal halide catalyst by drying a resulting filtrate.

2. The method according to claim 1, wherein the production waste liquid comprises about 1 wt % to about 10 wt % of the metal halide catalyst, about 60 wt % to about 95 wt % of the ethylene carbonate, and about 1 wt % to about 30 wt % of the by-products and impurities based on the total weight of the production waste liquid.

3. The method according to claim 1, wherein vacuum distillation of the production waste liquid is performed at a temperature of about 100° C. to about 150° C. and at a pressure of about 0.5 torr to about 20 torr.

4. The method according to claim 1, wherein the separated and recovered ethylene carbonate has a purity of about 99.5% or more, as calculated from an area of a peak detected by a flame ionization detector through a procedure in which a sample, prepared by dissolving the recovered ethylene carbonate in methanol to a concentration of 5 wt %, is injected into a gas chromatograph equipped with a DB-WAX column at an injection temperature of 250° C., followed by maintaining a chamber at a temperature of 40° C. for 5 minutes and raising the temperature of the chamber to 230° C. at a rate of 10° C./min.

5. The method according to claim 1, wherein the dichloromethane is added in an amount of about 80 parts by weight to about 500 parts by weight relative to about 100 parts by weight of the solid residue remaining after separation of the ethylene carbonate.

6. The method according to claim 1, wherein the water is added in an amount of about 80 parts by weight to about 500 parts by weight relative to about 100 parts by weight of the filtered solid residue remaining after removal of the by-products.

7. The method according to claim 1, wherein drying of the filtrate is performed at a temperature of about 60° C. to about 110° C. and at a pressure of about 1 torr to about 300 torr.

8. The method according to claim 1, wherein the recovered metal halide catalyst is free of detectable organic matter, including ethylene carbonate, as measured using a flame ionization detector through a procedure in which a sample, prepared by dissolving the recovered metal halide catalyst in methanol to a concentration of 5 wt %, is injected into a gas chromatograph equipped with a DB-WAX column at an injection temperature of 250° C., followed by maintaining a chamber at a temperature of 40° C. for 5 minutes and raising the temperature of the chamber to 230° C. at a rate of 10° C./min.

9. The method according to claim 1, wherein the method has a recovery rate of about 70% or more for the metal halide catalyst, as calculated according to Equation 1:
Metal halide catalyst recovery rate (%)=(Amount of recovered metal halide catalyst (g)/Amount of metal halide catalyst in ethylene carbonate production waste liquid (g))×100 [Equation 1]
where the amount of the metal halide catalyst in the ethylene carbonate production waste liquid is obtained by multiplying a weight of a production waste liquid used by a percentage content (%) of the metal halide catalyst in an entire production waste liquid.
